(19) United States
(12) Patent Application Publication
(10) Pub. No.: US 2023/0355327 A1
(43) Pub. Date: Nov. 9, 2023

(54) LAPAROSCOPIC SURGICAL ROBOTIC SYSTEM WITH INTERNAL DEGREES OF FREEDOM OF ARTICULATION

(71) Applicant: Vicarious Surgical Inc., Waltham, MA (US)

(72) Inventors: Sammy Khalifa, Medford, MA (US); Adam Sachs, Cambridge, MA (US)

(21) Appl. No.: 18/139,284

(22) Filed: Apr. 25, 2023

Related U.S. Application Data
(63) Continuation of application No. PCT/US2021/056912, filed on Oct. 27, 2021.
(60) Provisional application No. 63/106,688, filed on Oct. 28, 2020.

(51) Int. Cl. A61B 34/30; A61B 90/00

(52) U.S. Cl. CPC A61B 34/30; A61B 90/37; A61B 2034/305; A61B 2034/302; A61B 2090/371

(57) ABSTRACT

Systems, methods, and media for performing laparoscopic surgery with degrees of freedom of articulation that are internal to the body cavity of the subject during one or more laparoscopic surgical operations are provided. A system for performing laparoscopic surgery may comprise a robotic arm comprising one or more wrist elements. The one or more wrist elements may be configured to be inserted into a body cavity of a subject to perform one or more laparoscopic surgical operations therein. The robotic arm may be configured to provide one or more degrees of freedom of articulation that are internal to the body cavity of the subject during the one or more laparoscopic surgical operations.

### CROSS-REFERENCE

This application is a continuation application of International Application No. PCT/US2021/056912 filed Oct. 27, 2021, which claims the benefit of U.S. Provisional Application No. 63/106,688, filed Oct. 28, 2020, each of which is incorporated by reference herein in its entirety.

### BACKGROUND

Surgeons may perform laparoscopic surgery by creating one or more small incisions in a patient's body cavity (e.g., abdomen), through which small surgical tools and a camera may be inserted in order to perform a surgical procedure. Such minimally invasive surgery techniques may have advantages over non-laparoscopic surgery, such as reduced pain, reduced blood loss, reduced scarring, reduced follow-up care and hospital stays, and faster recovery times. Laparoscopic surgery may be performed using surgical robots, which use computer controls to manipulate surgical instruments and a camera, thereby providing increased precision and/or range of motion and/or vision.

### SUMMARY

Provided herein is a system for performing laparoscopic surgery, comprising: a set of robotic arms, each of the set of robotic arms comprising at least one end-effector, and at least one camera, wherein the at least one internal end-effector and the at least one camera have sufficient degrees of freedom of adjustment of position and sufficient degrees of freedom of adjustment of orientation to provide a full range of motion and orientation of operation and view perspective for performing the laparoscopic surgery while inserted into a body cavity (e.g., abdomen) of a subject. In some embodiments, the full range of motion and orientation of operation and view perspective comprises a front-facing, back-facing, side-facing, up-facing, down-facing, left-facing, or right-facing direction of motion or orientation of operation and view perspective, or any direction of motion or orientation of operation and view perspective therebetween. In some embodiments, the full range of motion and orientation of operation and view perspective comprises ability to be adjusted by 90 degrees between any two positions or directions of motion or orientation of operation and view perspective. In some embodiments, the system further comprises external degrees of freedom that enable internal degrees of freedom to be translated about the body cavity (e.g., abdomen) of the subject.

A robotic system of the present disclosure may comprise one or more anthropomorphic robotic arm instruments and one or more cameras (e.g., robotic cameras, which may work together as stereoscopic cameras such as actuatable stereoscopic cameras). In some embodiments, the robotic system may comprise two anthropomorphic robotic arm instruments and one actuatable stereoscopic camera. Each of the arm instruments and camera may be inserted one by one through the insertion site and trocar and into the patient's abdomen. Each instrument arm may have at least 1, 2, 3, 4, 5, 6, 7, 8, 9, 10, 11, 12, or more than 12 degrees of freedom internally (plus one for the end-effector). Further, the stereoscopic camera may have at least 1, 2, 3, 4, 5, 6, 7, 8, 9, 10, 11, 12, or more than 12 degrees of freedom internally. For example, seven degrees of freedom of the arm may be arranged in the following order starting proximally: a rotary actuator and hinge actuator that together mimic the motions of a human shoulder, a rotary actuator, a hinge actuator that mimics the motion of a human elbow, and a rotary actuator followed by two hinge-like actuators that together mimic the motion of a human wrist. Attached to the wrist may be an end-effector. Having many multiple degrees of freedom with a sufficient range of motion may enable the wrist of an arm to be able to reach a large number of possible positions at a large number of possible orientations (e.g., in front, in back, on both sides, up and down, left and right, etc.), thereby allowing a user of the surgical robotic system to be able to view and/or operate the surgical robotic system at any position and at any view orientation during a surgical operation (e.g., laparoscopic operation) inside a patient's body cavity (e.g., abdomen).

In some embodiments, each of the set of robotic arms comprises: a first shaft having a first axis of symmetry; a second shaft having a second axis of symmetry; a third shaft having a third axis of symmetry; an end effector for insertion into a body cavity of a subject to perform a laparoscopic surgical operation therein; a first actuator rotating the second shaft with respect to the first shaft about a first primary axis; a second actuator rotating the second shaft with respect to the second shaft about a second primary axis; and a third actuator rotating the end effector with respect to the third shaft about a third primary axis.

In some embodiments, the set of robotic arms provides at least one degree of freedom of articulation that is internal to the body cavity of the subject during the laparoscopic surgical operation. In some embodiments, the set of robotic arms provides at least 2, 3, 4, 5, 6, 7, 8, 9, 10, 11, 12, or more than 12 degrees of freedom of articulation that are internal to the body cavity of the subject during the laparoscopic surgical operation. In some embodiments, the system further comprises at least one camera for insertion into the body cavity of the subject to visualize the laparoscopic surgical operation. In some embodiments, the system further comprises a camera positioning arm, and a camera actuator rotating the at least one camera with respect to the camera positioning arm about a primary camera axis. In some embodiments, the at least one camera is configured to provide at least one degree of freedom of articulation that is internal to the body cavity of the subject during the laparoscopic surgical operation. In some embodiments, the at least one camera is configured to provide at least 2, 3, 4, 5, 6, 7, 8, 9, 10, 11, 12, or more than 12 degrees of freedom of articulation that are internal to the body cavity of the subject during the laparoscopic surgical operation. In some embodiments, the at least one camera comprise at least one stereoscopic camera. In some embodiments, the at least one stereoscopic camera comprises at least one actuatable stereoscopic camera. In some embodiments, the system provides at least 2, 3, 4, 5, 6, 7, 8, 9, 10, 11, 12, or more than 12 degrees of freedom of articulation that are internal to the body cavity of the subject selected from: an insertion degree of freedom, a roll degree of freedom, a pitch degree of freedom, and a yaw degree of freedom. In some embodiments, the camera comprises a flash, a lens, a flash, a light, or any combination thereof. In some embodiments, the camera comprises a stereoscopic camera, an infrared camera, an optical camera, or any combination thereof. In some embodiments, the end effector is a pincer, a grasper, a needle driver, a forceps, or any combination thereof. In some embodiments, the camera actuator further rotates the camera with respect to the camera positioning arm about a secondary camera axis that is perpendicular to the primary camera axis. In some embodiments, the primary camera axis and the secondary camera axis are perpendicular to an axis of symmetry of the camera positioning arm. In some embodiments, the first actuator further rotates the second shaft with respect to the first shaft about a first secondary axis perpendicular to the first primary axis. In some embodiments, the second actuator further rotates the third shaft with respect to the second shaft about a second secondary axis perpendicular to the second primary axis. In some embodiments, the third actuator further rotates the end effector with respect to the third shaft about a third secondary axis perpendicular to the third primary axis. In some embodiments, the system further comprises a trocar, and wherein the camera positioning arm, the first shaft of one or more of the two or more arms, or both are translatable with respect to the trocar.

Another aspect provided herein is a method for performing laparoscopic surgery, comprising inserting at least a portion of a set of robotic arms into a body cavity of a subject to perform a laparoscopic surgical operation therein, each of the set of robotic arms comprising at least one end-effector, and at least one camera, wherein the at least one internal end-effector and the at least one camera have sufficient degrees of freedom of adjustment of position and sufficient degrees of freedom of adjustment of orientation to provide a full range of motion and orientation of operation and view perspective for performing the laparoscopic surgery while inserted into a body cavity of a subject.

In some embodiments, the body cavity is an abdomen of the subject. In some embodiments, the full range of motion and orientation of operation and view perspective comprises a front-facing, back-facing, side-facing, up-facing, down-facing, left-facing, or right-facing direction of motion or orientation of operation and view perspective, or any direction of motion or orientation of operation and view perspective therebetween. In some embodiments, the full range of motion and orientation of operation and view perspective comprises ability to be adjusted by 90 degrees between any two positions or directions of motion or orientation of operation and view perspective. In some embodiments, the set of robotic arms and/or the at least one camera comprise external degrees of freedom that enable internal degrees of freedom to be translated about the body cavity of the subject.

In some embodiments, each of the set of robotic arms comprises: a first shaft having a first axis of symmetry; a second shaft having a second axis of symmetry; a third shaft having a third axis of symmetry; an end effector for insertion into a body cavity of a subject to perform a laparoscopic surgical operation therein; a first actuator rotating the second shaft with respect to the first shaft about a first primary axis; a second actuator rotating the second shaft with respect to the second shaft about a second primary axis; and a third actuator rotating the end effector with respect to the third shaft about a third primary axis.

In some embodiments, the set of robotic arms comprises at least two robotic arms. In some embodiments, the set of robotic arms provides at least one degree of freedom of articulation that is internal to the body cavity of the subject during the laparoscopic surgical operation. In some embodiments, the set of robotic arms provides at least 2, 3, 4, 5, 6, 7, 8, 9, 10, 11, 12, or more than 12 degrees of freedom of articulation that are internal to the body cavity of the subject during the laparoscopic surgical operation. In some embodiments, the set of robotic arms further comprises at least one camera for insertion into the body cavity of the subject to visualize the laparoscopic surgical operation. In some embodiments, the set of robotic arms further comprises a camera positioning arm, and a camera actuator rotating the at least one camera with respect to the camera positioning arm about a primary camera axis. In some embodiments, the at least one camera is configured to provide at least one degree of freedom of articulation that is internal to the body cavity of the subject during the laparoscopic surgical operation. In some embodiments, the at least one camera is configured to provide at least 2, 3, 4, 5, 6, 7, 8, 9, 10, 11, 12, or more than 12 degrees of freedom of articulation that are internal to the body cavity of the subject during the laparoscopic surgical operation. In some embodiments, the at least one camera comprise at least one stereoscopic camera. In some embodiments, the at least one stereoscopic camera comprises at least one actuatable stereoscopic camera. In some embodiments, the system provides at least 2, 3, 4, 5, 6, 7, 8, 9, 10, 11, 12, or more than 12 degrees of freedom of articulation that are internal to the body cavity of the subject selected from: an insertion degree of freedom, a roll degree of freedom, a pitch degree of freedom, and a yaw degree of freedom. In some embodiments, the camera comprises a flash, a lens, a flash, a light, or any combination thereof. In some embodiments, the camera comprises a stereoscopic camera, an infrared camera, an optical camera, or any combination thereof. In some embodiments, the end effector is a pincer, a grasper, a needle driver, a forceps, or any combination thereof. In some embodiments, the camera actuator further rotates the camera with respect to the camera positioning arm about a secondary camera axis that is perpendicular to the primary camera axis. In some embodiments, the primary camera axis and the secondary camera axis are perpendicular to an axis of symmetry of the camera positioning arm. In some embodiments, the first actuator further rotates the second shaft with respect to the first shaft about a first secondary axis perpendicular to the first primary axis. In some embodiments, the second actuator further rotates the third shaft with respect to the second shaft about a second secondary axis perpendicular to the second primary axis. In some embodiments, the third actuator further rotates the end effector with respect to the third shaft about a third secondary axis perpendicular to the third primary axis. In some embodiments, the set of robotic arms further comprises a trocar, and wherein the camera positioning arm, the first shaft of one or more of the two or more arms, or both are translatable with respect to the trocar.

Another aspect provided herein is a platform comprising: the system herein, a motor providing power to the system; and a gantry coupled to the motor.

In some embodiments, the motor provides power to one or more of: the camera actuator; the first actuator; the second actuator; and the third actuator. In some embodiments, the gantry couples to the motor by one or more of a rotatable coupling and a translatable coupling. In some embodiments, the platform further comprises a surgical table. In some embodiments, the platform further comprises a display receiving an image from the camera. In some embodiments, the display is a head-mounted display. In some embodiments, the platform further comprises an input providing an actuation command to the motor.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent application file contains at least one drawing executed in color. Copies of this patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows one example of a robotic arm comprising positioning elements, wrist elements, a support tube, and a trocar.

FIG. 2 shows one example of a robotic arm with 8 degrees of freedom, comprising positioning elements (including joints J0, J1, J2, and J3) and wrist elements (including joints J4, J5, J6, and J7).

FIG. 3 shows one example of a robotic arm configured for 360-degree visualization and reach, which is able to look and operate in a sphere from a single incision.

FIGS. 4A-4C show one example of a robotic arm configured to operate front and side-to-side, as illustrated by a right-facing operating configuration (FIG. 4A), a front-facing operating configuration (FIG. 4B), and a left-facing operating configuration (FIG. 4C).

FIGS. 5A-5C show one example of a robotic arm configured to operate front, up, down, and backwards, as illustrated by an up-facing operating configuration (FIG. 5A), a front-facing operating configuration (FIG. 5B), and a down-facing operating configuration (FIG. 5C).

FIGS. 6A-6C show one example of a robotic arm configured to operate up and backwards, as illustrated by an up-facing operating configuration (FIG. 6A), a transition from an up-facing to a backwards-facing operating configuration (FIG. 6B), and a backwards-facing operating configuration (FIG. 6C).

FIG. 7 shows one example of a robot support system (RSS) comprising axes and translating positioning elements about an abdomen of a subject.

FIGS. 8A-8D show one example of various axes of the RSS, including a side view of an insertion axis (FIG. 8A), a side view of a roll axis (FIG. 8B), a side view of a pitch axis (FIG. 8C), and a top view of a yaw axis (FIG. 8D).

FIGS. 9A-9B show one example of using a robotic arm to approach a surgical target from two sides (left side: FIG. 9A; right side: FIG. 9B) by adjusting the yaw and insertion axes as well as relative positions of positioning elements. These figures illustrate front and side facing views of the robotic arm relative to a trocar.

FIGS. 10A-10B show one example of using a robotic arm to approach a surgical target from two sides (left side: FIG. 10A; right side: FIG. 10B) by adjusting the yaw and insertion axes as well as relative positions of positioning elements. These figures illustrate backwards-facing views (toward an incision) of the robotic arm relative to a trocar.

FIGS. 11A-11D show one example of using a robotic arm to traverse an abdomen of a subject from a single incision, by adjusting the yaw and insertion axes as well as relative positions of some of the positioning elements. These figures illustrate front and side facing views of the robotic arm relative to a trocar.

FIG. 12 shows one example of a computer system 1201 that is programmed or otherwise configured to direct operation of a device or system as described herein, including movement of components of the device or system and/or performing a surgical procedure using the device or system.

### DETAILED DESCRIPTION

Laparoscopic surgery (e.g., manual laparoscopic surgery and robotic laparoscopic surgery) may encounter significant challenges in ensuring proper placement of the incisions and the trocars relative to the subject's (e.g., patient's) body cavity (e.g., abdomen), through which instruments are inserted and surgical materials are exchanged. For example, if a surgeon desires to operate on one side of a patient's abdomen, typically the incision may be placed through the opposite side of the abdomen. This may be due to the straight-stick nature of laparoscopic instruments and surgical cameras. Additionally, if the surgeon desires to operate on a subsequent area within the abdomen that is not immediately adjacent to the first area, then additional incisions may need to be created to accommodate the new surgical site. Even with the wristed straight-stick instruments of robotic laparoscopic surgery and the adjustability of most laparoscopes, the location of the incision and the trocar may directly affect and limit the locations where a surgeon is able to work and visualize.

These limitations of laparoscopic instruments and cameras may arise because of the fact that the majority of the articulation and degrees of freedom of articulation may occur external to the patient's abdomen. Each instrument (e.g., the camera) may be articulated externally with large, powerful actuators, and may then pass through a fulcrum (e.g., the incision site), both of which place limits on the articulation that is possible inside the patient.

In light of these challenges, there exists a need for improved systems and methods of laparoscopic surgery that offer increased degrees of freedom of articulation, thereby decreasing limitations and challenges in ensuring proper placement of incisions and trocars in a patient's abdomen. Recognizing the need for improved systems and methods of laparoscopic surgery that offer increased degrees of freedom of articulation, the present disclosure provides systems and methods for robotic surgery that provide degrees of freedom of articulation that are internal to a patient's body cavity (e.g., abdomen). By inserting a surgical robot with sufficient degrees of freedom of articulation and range of motion in the robotic arms within the patient's abdomen as well as using a visualization system with sufficient degrees of freedom of articulation and range of motion within the patient's abdomen, improved modalities of robotic surgery are enabled, advantageously providing the surgeon with full freedom to adjust the locations on the patient's abdomen where he or she is working and looking within a full spherical envelope.

While various embodiments are shown and described herein, it will be obvious to those skilled in the art that such embodiments are provided by way of example only. It should be understood that various alternatives to the embodiments herein are employed.

As used herein, the singular forms “a”, “an”, and “the” include plural references unless the context clearly dictates otherwise. Any reference to “or” herein is intended to encompass “and/or” unless otherwise stated.

Surgeons may perform laparoscopic surgery by creating one or more small incisions in a patient's body cavity (e.g., abdomen), through which small surgical tools and a camera may be inserted in order to perform a surgical procedure. Such minimally invasive surgery techniques may have advantages over non-laparoscopic surgery, such as reduced pain, reduced blood loss, reduced scarring, reduced follow-up care and hospital stays, and faster recovery times. Laparoscopic surgery may be performed using surgical robots, which use computer controls to manipulate surgical instruments and a camera, thereby providing increased precision and/or range of motion and/or vision.

Laparoscopic surgery (e.g., manual laparoscopic surgery and robotic laparoscopic surgery) may encounter significant challenges in ensuring proper placement of the incisions and the trocars relative to the subject's (e.g., patient's) body cavity (e.g., abdomen), through which instruments are inserted and surgical materials are exchanged. For example, if a surgeon desires to operate on one side of a patient's abdomen, typically the incision may be placed through the opposite side of the abdomen. This may be due to the straight-stick nature of laparoscopic instruments and surgical cameras. Additionally, if the surgeon desires to operate on a subsequent area within the abdomen that is not immediately adjacent to the first area, then additional incisions may need to be created to accommodate the new surgical site. Even with the wristed straight-stick instruments of robotic laparoscopic surgery and the adjustability of most laparoscopes, the location of the incision and the trocar may directly affect and limit the locations where a surgeon is able to work and visualize.

These limitations of laparoscopic instruments and cameras may arise because of the fact that the majority of the articulation and degrees of freedom of articulation may occur external to the patient's abdomen. Each instrument (e.g., the camera) may be articulated externally with large, powerful actuators, and may then pass through a fulcrum (e.g., the incision site), both of which place limits on the articulation that is possible inside the patient.

In light of these challenges, there exists a need for improved systems and methods of laparoscopic surgery that offer increased degrees of freedom of articulation, thereby decreasing limitations and challenges in ensuring proper placement of incisions and trocars in a patient's abdomen. Recognizing the need for improved systems and methods of laparoscopic surgery that offer increased degrees of freedom of articulation, the present disclosure provides systems and methods for robotic surgery that provide degrees of freedom of articulation that are internal to a patient's body cavity (e.g., abdomen). By inserting a surgical robot with sufficient degrees of freedom of articulation and range of motion in the robotic arms within the patient's abdomen as well as using a visualization system with sufficient degrees of freedom of articulation and range of motion within the patient's abdomen, improved modalities of robotic surgery are enabled, advantageously providing the surgeon with full freedom to adjust the locations on the patient's abdomen where he or she is working and looking within a full spherical envelope.

In an aspect, the present disclosure provides a system for performing laparoscopic surgery, comprising: a robotic arm comprising one or more wrist elements, wherein the one or more wrist elements are configured to be inserted into a body cavity of a subject to perform one or more laparoscopic surgical operations therein, wherein the robotic arm is configured to provide one or more degrees of freedom of articulation that are internal to the body cavity of the subject during the one or more laparoscopic surgical operations.

In some embodiments, the robotic arm is configured to provide at least 2, 3, 4, 5, 6, 7, 8, 9, 10, 11, 12, or more than 12 degrees of freedom of articulation that are internal to the body cavity of the subject during the one or more laparoscopic surgical operations. In some embodiments, the robotic arm is configured to provide one or more degrees of freedom of articulation that are internal to the body cavity of the subject using one or more of: a rotary actuator and hinge actuator that together mimic the motions of a human shoulder, a rotary actuator, a hinge actuator that mimics the motion of a human elbow, and a rotary actuator followed by two hinge-like actuators that together mimic the motion of a human wrist.

In some embodiments, the system further comprises one or more cameras (e.g., robotic cameras) configured to be inserted into the body cavity of the subject to visualize the one or more laparoscopic surgical operations. In some embodiments, the one or more cameras are configured to provide one or more degrees of freedom of articulation that are internal to the body cavity of the subject during the one or more laparoscopic surgical operations. In some embodiments, the one or more cameras are configured to provide at least 2, 3, 4, 5, 6, 7, 8, 9, 10, 11, 12, or more than 12 degrees of freedom of articulation that are internal to the body cavity of the subject during the one or more laparoscopic surgical operations. In some embodiments, the one or more cameras (e.g., robotic cameras) are configured to provide one or more degrees of freedom of articulation that are internal to the body cavity of the subject using one or more of: a rotary actuator and a hinge-like actuator. In some embodiments, the one or more cameras comprise one or more camera modules or sensors (e.g., stereoscopic cameras). In some embodiments, the one or more camera modules or sensors comprise actuatable stereoscopic cameras. In some embodiments, the one or more camera modules or sensors can work together to form one or more stereoscopic cameras.

In some embodiments, the one or more wrist elements are configured to provide one or more degrees of freedom of articulation that are internal to the body cavity of the subject during the one or more laparoscopic surgical operations. In some embodiments, the one or more wrist elements are configured to provide at least 2, 3, 4, 5, 6, 7, 8, 9, 10, 11, 12, or more than 12 degrees of freedom of articulation that are internal to the body cavity of the subject during the one or more laparoscopic surgical operations.

In some embodiments, the robotic arm comprises one or more positioning elements configured to allow control of the positioning of the one or more wrist elements. In some embodiments, the one or more positioning elements are configured to provide one or more degrees of freedom of articulation that are internal to the body cavity of the subject during the one or more laparoscopic surgical operations. In some embodiments, the one or more positioning elements are configured to provide at least 2, 3, 4, 5, 6, 7, 8, 9, 10, 11, 12, or more than 12 degrees of freedom of articulation that are internal to the body cavity of the subject during the one or more laparoscopic surgical operations. In some embodiments, the one or more positioning elements are configured to provide at least 4 degrees of freedom of articulation that are internal to the body cavity of the subject during the one or more laparoscopic surgical operations.

In some embodiments, at least one of the robotic arm, the one or more wrist elements, the one or more positioning elements, the one or more cameras (e.g., robotic cameras) are configured to provide degrees of freedom of articulation that are internal to the body cavity of the subject selected from: an insertion degree of freedom, a roll degree of freedom, a pitch degree of freedom, and a yaw degree of freedom.

In another aspect, the present disclosure provides a method for performing laparoscopic surgery, comprising: inserting one or more wrist elements of a robotic arm into a body cavity of a subject to perform one or more laparoscopic surgical operations therein, wherein the robotic arm is configured to provide one or more degrees of freedom of articulation that are internal to the body cavity of the subject during the one or more laparoscopic surgical operations.

In some embodiments, the robotic arm is configured to provide at least 2, 3, 4, 5, 6, 7, 8, 9, 10, 11, 12, or more than 12 degrees of freedom of articulation that are internal to the body cavity of the subject during the one or more laparoscopic surgical operations. In some embodiments, the robotic arm is configured to provide one or more degrees of freedom of articulation that are internal to the body cavity of the subject using one or more of: a rotary actuator and hinge actuator that together mimic the motions of a human shoulder, a rotary actuator, a hinge actuator that mimics the motion of a human elbow, and a rotary actuator followed by two hinge-like actuators that together mimic the motion of a human wrist.

In some embodiments, the method further comprises inserting one or more cameras (e.g., robotic cameras) into the body cavity of the subject to visualize the one or more laparoscopic surgical operations. In some embodiments, the one or more cameras are configured to provide one or more degrees of freedom of articulation that are internal to the body cavity of the subject during the one or more laparoscopic surgical operations. In some embodiments, the one or more cameras are configured to provide at least 2, 3, 4, 5, 6, 7, 8, 9, 10, 11, 12, or more than 12 degrees of freedom of articulation that are internal to the body cavity of the subject during the one or more laparoscopic surgical operations. In some embodiments, the one or more cameras are configured to provide one or more degrees of freedom of articulation that are internal to the body cavity of the subject using one or more of: a rotary actuator and a hinge-like actuator. In some embodiments, the one or more cameras comprise one or more stereoscopic cameras. In some embodiments, the one or more stereoscopic cameras comprise actuatable stereoscopic cameras.

In some embodiments, the one or more wrist elements are configured to provide one or more degrees of freedom of articulation that are internal to the body cavity of the subject during the one or more laparoscopic surgical operations. In some embodiments, the one or more wrist elements are configured to provide at least 2, 3, 4, 5, 6, 7, 8, 9, 10, 11, 12, or more than 12 degrees of freedom of articulation that are internal to the body cavity of the subject during the one or more laparoscopic surgical operations.

In some embodiments, the robotic arm comprises one or more positioning elements configured to allow control of the positioning of the one or more wrist elements. In some embodiments, the one or more positioning elements are configured to provide one or more degrees of freedom of articulation that are internal to the body cavity of the subject during the one or more laparoscopic surgical operations. In some embodiments, the one or more positioning elements are configured to provide at least 2, 3, 4, 5, 6, 7, 8, 9, 10, 11, 12, or more than 12 degrees of freedom of articulation that are internal to the body cavity of the subject during the one or more laparoscopic surgical operations. In some embodiments, the one or more positioning elements are configured to provide at least 4 degrees of freedom of articulation that are internal to the body cavity of the subject during the one or more laparoscopic surgical operations.

In some embodiments, at least one of the robotic arm, the one or more wrist elements, the one or more positioning elements, the one or more cameras (e.g., robotic cameras) is configured to provide degrees of freedom of articulation that are internal to the body cavity of the subject selected from: an insertion degree of freedom, a roll degree of freedom, a pitch degree of freedom, and a yaw degree of freedom.

In another aspect, the present disclosure provides a non-transitory computer-readable medium comprising machine-executable code that, upon execution by one or more computer processors, implements a method for performing laparoscopic surgery, the method comprising: controlling one or more wrist elements of a robotic arm to be inserted into a body cavity of a subject to perform one or more laparoscopic surgical operations therein, wherein the robotic arm is configured to provide one or more degrees of freedom of articulation that are internal to the body cavity of the subject during the one or more laparoscopic surgical operations.

Also described herein is a non-transitory computer-readable medium comprising machine-executable code that, upon execution by one or more computer processors, implements any of the methods above or elsewhere herein.

Also described herein is a system comprising one or more computer processors and computer memory coupled thereto. The computer memory comprises machine executable code that, upon execution by the one or more computer processors, implements any of the methods above or elsewhere herein.

A robotic system of the present disclosure may comprise one or more anthropomorphic robotic arm instruments and one or more cameras (e.g., robotic cameras, which may work together as stereoscopic cameras such as actuatable stereoscopic cameras). In some embodiments, the robotic system may comprise two anthropomorphic robotic arm instruments and one actuatable stereoscopic camera. Each of the arm instruments and camera may be inserted one by one through the insertion site and trocar and into the patient's abdomen. Each instrument arm may have at least 1, 2, 3, 4, 5, 6, 7, 8, 9, 10, 11, 12, or more than 12 degrees of freedom internally (plus one for the end-effector). Further, the stereoscopic camera may have at least 1, 2, 3, 4, 5, 6, 7, 8, 9, 10, 11, 12, or more than 12 degrees of freedom internally. For example, seven degrees of freedom of the arm may be arranged in the following order starting proximally: a rotary actuator and hinge actuator that together mimic the motions of a human shoulder, a rotary actuator, a hinge actuator that mimics the motion of a human elbow, and a rotary actuator followed by two hinge-like actuators that together mimic the motion of a human wrist. Attached to the wrist may be an end-effector. Having many multiple degrees of freedom with a sufficient range of motion may enable the wrist of an arm to be able to reach a large number of possible positions at a large number of possible orientations (e.g., in front, in back, on both sides, up and down, left and right, etc.), thereby allowing a user of the surgical robotic system to be able to view and/or operate the surgical robotic system at any position and at any view orientation during a surgical operation (e.g., laparoscopic operation) inside a patient's body cavity (e.g., abdomen). Further, by using cameras with at least 3 degrees of freedom, each with a sufficient range of motion, the cameras may be able to adjust its view in any direction (e.g., in front, in back, on both sides, up and down, left and right, etc.), and adjust the horizon of the image for the viewer. In contrast, a camera with only 2 degrees of freedom may be able to adjust its view in any direction, but lack ability to adjust horizon.

In some embodiments, the degrees of freedom of the camera are arranged in the following order starting proximally: rotary actuator, hinge-like actuator and a rotary actuator. Together, these internal degrees of freedom enable the surgeon to adjust the working site of the robot at his or her discretion, up to and including close proximity to the insertion site and trocar. Therefore, systems and methods of the present disclosure provide surgical robotic approaches that enable unprecedented flexibility and capability in laparoscopic surgery.

The robotic arms can comprise one or more positioning elements and wrist elements, both of which may be necessary to enable high-dexterity surgical manipulation anywhere in the patient's abdomen from a single incision. The positioning elements may comprise a system with at least 4 degrees of freedom. The purpose of these positioning elements may be to enable the end-effector to be positioned anywhere relative to the incision site and to roughly orient the end-effector. Having greater than 4 degrees of freedom allows more flexibility in positioning of the end-effector and finer orientability of the end-effector.

In some embodiments, the robotic arm comprises a support tube coupled to a proximal rotary joint, which is then coupled to a distal hinge joint, which is then coupled to a distal rotary joint, which is then coupled to a distal hinge joint. This configuration of robotic arm may allow the robotic arm to have a high degree of freedom of position and orientation around the incision site.

In some embodiments, the robotic arm comprises a fifth joint distal to the fourth, that comprises a rotary joint that allows for the roll of the end-effector to be adjusted.

In some embodiments, the robotic arm comprises a support tube coupled to a proximal rotary joint, which is then distally coupled to a hinge joint which is then distally coupled to a hinge joint orthogonal to the previous joint, which is then distally coupled to a hinge joint orthogonal to the previous joint.

In some embodiments, the robotic arm comprises a support tube coupled to a proximal rotary joint, which is then distally coupled to a ball joint (having 2 degrees of freedom), which is then distally coupled to a hinge joint or another ball joint.

In some embodiments, the robotic arm comprises a support tube coupled to a proximal rotary joint, which is then coupled to a snake robot with more than 3 degrees of freedom.

In some embodiments, the rotary joint is external to the patient, and the rotation is transferred to the internal joints by the support tube via the incision site. These embodiments may enable the operator to position and roughly orient the end-effector to work relative to the trocar forward, up, down, left, right, and back at the incision site. The maneuverability of the system may be limited by the link lengths and the range of motion of each joint, and may manifest itself as there being a region that is too close to position the robot and another region that is too far to position the robot in.

These limits to the positionability of the robot may be augmented and mitigated by adding additional external degrees of freedom that move the positioning elements relative to the patient and surgical site from outside the patient. These degrees of freedom may be similar to those used in certain approaches to manual laparoscopic surgery or in some robotic surgeries, and enable rough and large adjustments of the position of the positioning elements within the patient's anatomy. Some of the possible degrees of freedom are as follows: insertion, roll, yaw, and pitch about the incision site.

The insertion degree of freedom may be a linear translation of the positioning elements via the support tube along the lengthwise axis of the trocar, either deeper into the patient or retracted away from the patient. This may enable the positioning elements to traverse from one site of the surgical site to the other.

The roll degree of freedom may be a rotation of the positioning elements via the support tube about the lengthwise axis of the trocar (or another parallel axis). This may enable the orientation of the positioning elements to be adjusted to the operator's comfort of desire.

The yaw degree of freedom may be a rotation of the positioning elements via the support tube about an axis perpendicular to the lengthwise axis of the trocar and typically perpendicular to the ground. This may enable the positioning elements to traverse left and right (relative to the trocar) and to slightly adjust their orientation.

The pitch degree of freedom may be a rotation of the positioning elements via the support tube about an axis perpendicular to the lengthwise axis of the trocar and typically parallel to the ground. This may enable the positioning elements to traverse up and down (relative to the trocar) and to slightly adjust their orientation. As in both manual and robotic laparoscopy, both the yaw and pitch degrees of freedom may involve a rotation of the trocar relative to the patient, resulting in some temporary stretching of the patient's abdominal wall and surrounding tissue.

In some embodiments, the axes for the yaw, pitch, and roll degrees of freedom all pass through a single point located somewhere along the lengthwise axis of the trocar, which may be referred to as the trocar pivot point or virtual center. In this embodiment, that single point is located in the middle of the patient's abdominal wall such that the magnitude of the temporary stretching of the patient's tissue is minimized.

In some embodiments, the external degrees of freedom comprise only yaw, pitch, and insertion. In this embodiment, the lack of roll may be compensated for with degrees of freedom of the positioning elements of the system with some cost to the internal positionability of the end-effector.

In some embodiments, the external degrees of freedom comprise only yaw and insertion. In this embodiment, the lack of roll and pitch can be compensated for with degrees of freedom of the positioning elements of the system with some cost to the internal positionability of the end-effector.

On the distal end of the position elements, the wrist elements may be situated. The wrist elements may comprise an end-effector and at least two degrees of freedom. The wrist elements may enable fine control of the orientation of the end-effector and power the end-effector. To enable the finest adjustability of orientation and the highest dexterity, the axes of the two degrees of freedom may be in close proximity to each other and perpendicular to each other. The larger the distance between the two axes, the more translation that occurs when trying to adjust the orientation of the wrist. This is translation that is typically undesired and must be compensated for by the positioning elements, resulting in overall degraded motion quality and dexterity.

In some embodiments, the axes of the two degrees of freedom may be collocated in order to minimize the unwanted translation. In some embodiments, the wrist elements comprise a 2 degree-of-freedom ball joint, upon which is distally attached an end-effector. In some embodiments, the wrist elements comprise a hinge joint to which distally is coupled another hinge joint with an axis perpendicular to that of the proximal hinge, to which is distally coupled an end-effector.

In some embodiments, the robotic arm comprises a third more proximal joint comprising a rotary joint that provides for a rotation about its lengthwise axis. This additional rotational degree of freedom may enable the end-effector to roll about its lengthwise axis, similar to how pronation or supination of a human forearm allows for rolling of one's wrist or fingers. In some embodiments, this additional rotary joint augments positioning elements that are capable of providing some rotation about its lengthwise axis at its distal end to make the wrist (roll degree of freedom) have higher dexterity. In some embodiments, where the positioning elements are incapable of providing adequate control of rotation about the lengthwise axis at its distal end, the additional rotary joint enables high-dexterity control of the wrist roll.

In some embodiments, the end-effector comprises a set of jaws, the axes of which are collocated and that are designed to grip objects such as tissue, suture, or needles. In some embodiments, the end-effector is designed to deliver current to tissue as part of an electrocautery system. The end-effectors may comprise anything necessary to perform the required surgical procedure.

Systems of the present disclosure may advantageously combine a set of positioning elements, additional external degrees of freedom, and a set of wrist elements, thereby enabling movement about the entire abdominal cavity of a patient, such that the robotic arm is able to position and orient itself in any location desirable to the operator, in order to provide the high dexterity needed to successfully and easily perform the surgical procedure.

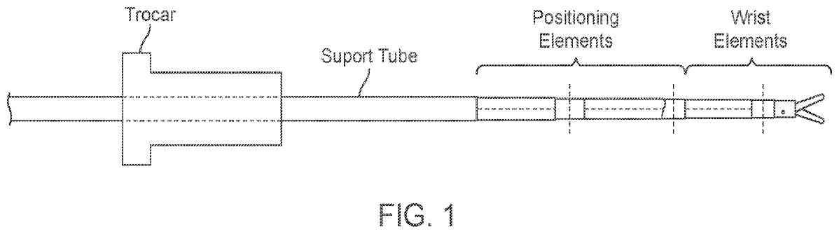

FIG. 1 shows one example of a robotic arm comprising positioning elements, wrist elements, a support tube, and a trocar.

### System for Performing Laparoscopic Surgery

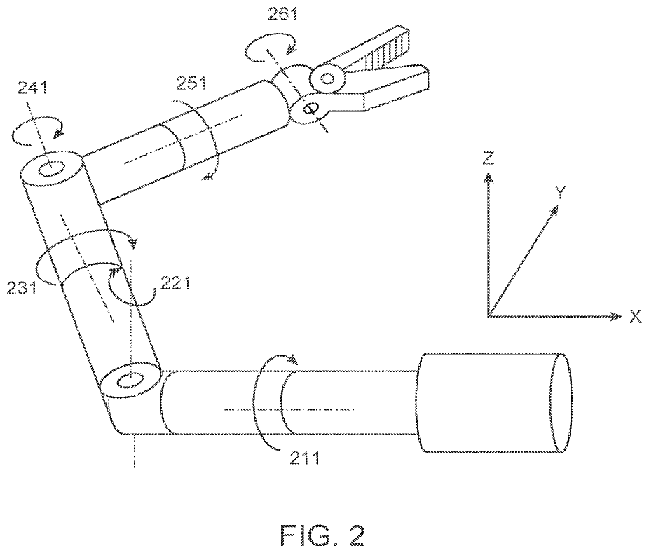

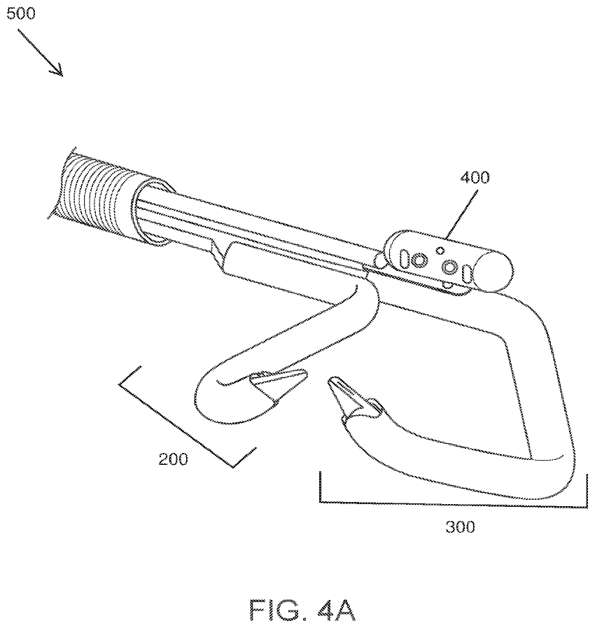

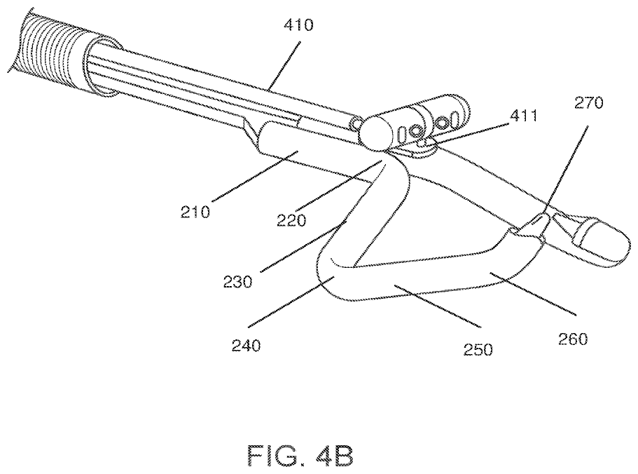

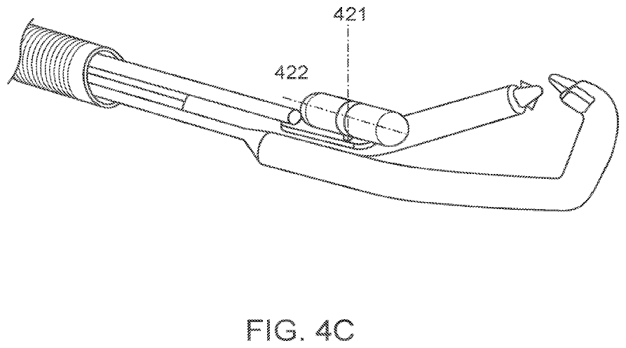

Provided herein, per FIGS. 2, 4A and 4B is a system for performing laparoscopic surgery 500. In some embodiments, the system 500 comprises a set of robotic arms 200 300. In some embodiments, the system 500 comprises a first robotic arm 200 and a second robotic arm 300.

In some embodiments, each of the set of robotic arms 200 300 comprises a first shaft 210, a first actuator 220, a second shaft 230, a second actuator 240, a third shaft 250, a third actuator 260, and an end effector 270. In some embodiments, the first shaft 210 has a first axis of symmetry 211. In some embodiments, the second shaft 230 has a second axis of symmetry 231. In some embodiments, the third shaft 250 has a third axis of symmetry 251.

In some embodiments, the first actuator 220, the second actuator 240, the third actuator 260, or any combination thereof actuate about 1 degree of freedom. In some embodiments, the first actuator 220, the second actuator 240, the third actuator 260, or any combination thereof actuate about 2 degrees of freedom. In some embodiments, the first actuator 220, the second actuator 240, the third actuator 260, or any combination thereof actuate about 3 degrees of freedom. In some embodiments, the first actuator 220, the second actuator 240, and the third actuator 260 are configured to be controlled in order to provide degrees of freedom of articulation sufficient to enable the system 500 provide surgical operations (e.g., cauterizing, clamping, cutting, manipulating tissue, suturing, making incisions, etc.) during a laparoscopic surgery.

In some embodiments, the first actuator 220 rotates the second shaft 230 with respect to the first shaft 210 about a first primary axis 221. In some embodiments, the second actuator 240 rotates the second shaft 230 with respect to the second shaft 230 about a second primary axis 241. In some embodiments, the third actuator 260 rotates the end effector 270 with respect to the third shaft 250 about a third primary axis 261. In some embodiments, the first actuator 220 further rotates the second shaft 230 with respect to the first shaft 210 about a first secondary axis perpendicular to the first primary axis 221. In some embodiments, the second actuator 240 further rotates the third shaft 250 with respect to the second shaft 230 about a second secondary axis perpendicular to the second primary axis 241. In some embodiments, the third actuator 260 further rotates the end effector 270 with respect to the third shaft 250 about a third secondary axis perpendicular to the third primary axis 261. In some embodiments, the system 500 further comprises a trocar, and wherein the camera 400 positioning arm, the first shaft 210 of one or more of the two or more arms 200 300, or both are translatable with respect to the trocar.

In some embodiments, the end effector 270 is configured for insertion into a body cavity of a subject to perform a laparoscopic surgical operation therein.

In some embodiments, the set of robotic arms 200 300 provides at least one degree of freedom of articulation that is internal to the body cavity of the subject during the laparoscopic surgical operation. In some embodiments, the set of robotic arms 200 300 provides at least 2, 3, 4, 5, 6, 7, 8, 9, 10, 11, 12, or more than 12 degrees of freedom of articulation that are internal to the body cavity of the subject during the laparoscopic surgical operation. In some embodiments, the system 500 provides at least 2, 3, 4, 5, 6, 7, 8, 9, 10, 11, 12, or more than 12 degrees of freedom of articulation that are internal to the body cavity of the subject selected from: an insertion degree of freedom, a roll degree of freedom, a pitch degree of freedom, and a yaw degree of freedom.

In some embodiments, the system 500 further comprises at least one camera 400 for insertion into the body cavity of the subject to visualize the laparoscopic surgical operation. In some embodiments, the system 500 further comprises a camera positioning arm 410, and a camera actuator 411 rotating the at least one camera 400 with respect to the camera positioning arm 410 about a primary camera axis. In some embodiments, the at least one camera 400 is configured to provide at least one degree of freedom of articulation that is internal to the body cavity of the subject during the laparoscopic surgical operation. In some embodiments, the at least one camera 400 is configured to provide at least 2, 3, 4, 5, 6, 7, 8, 9, 10, 11, 12, or more than 12 degrees of freedom of articulation that are internal to the body cavity of the subject during the laparoscopic surgical operation. In some embodiments, the at least one camera 400 comprises at least one stereoscopic camera. In some embodiments, the at least one stereoscopic camera comprises at least one actuatable stereoscopic camera. In some embodiments, the camera 400 comprises a flash, a lens, a flash, a light, or any combination thereof. In some embodiments, the camera 400 comprises a stereoscopic camera, an infrared camera, an optical camera, or any combination thereof. In some embodiments, the end effector 270 is a pincer, a grasper, a needle driver, a forceps, or any combination thereof. In some embodiments, the camera actuator 411 further rotates the camera 400 with respect to the camera 400 positioning arm about a secondary camera axis that is perpendicular to the primary camera axis. In some embodiments, the primary camera axis and the secondary camera axis are perpendicular to an axis of symmetry of the camera positioning arm 410.

In some embodiments, the robotic arm comprises a trocar, which may be a rigid medical device configured to be inserted into a body cavity (e.g., abdomen) of a subject during laparoscopic surgery. A trocar may comprise a sharp point (e.g., a sharp triangular point) configured to puncture the body cavity of the subject and/or to be inserted into the body cavity of the subject, thereby providing intra-abdominal access. Alternatively, the trocar may support wrist elements that comprise one or more sharp points (e.g., sharp triangular points) configured to puncture the body cavity of the subject and/or to be inserted into the body cavity of the subject, thereby providing intra-abdominal access.

In some embodiments, the robotic arm comprises a support tube, which may be inserted through the trocar (e.g., along a longitudinal axis of the trocar). The support tube may be configured to provide mechanical and structural support to the trocar and the positioning elements and wrist elements. The support tube may be configured to provide electrical power or electrical control signals (e.g., via electrical cables) to the positioning elements and wrist elements.

In some embodiments, the robotic arm comprises one or more positioning elements, which may be configured to position the wrist elements at a desired location during laparoscopic surgery. The positioning elements may be configured to provide sufficient degrees of freedom of articulation to the wrist elements.

In some embodiments, the robotic arm comprises one or more wrist elements, which may be configured to provide surgical operations such as cauterizing, clamping, cutting, manipulating tissue, suturing, making incisions, etc.

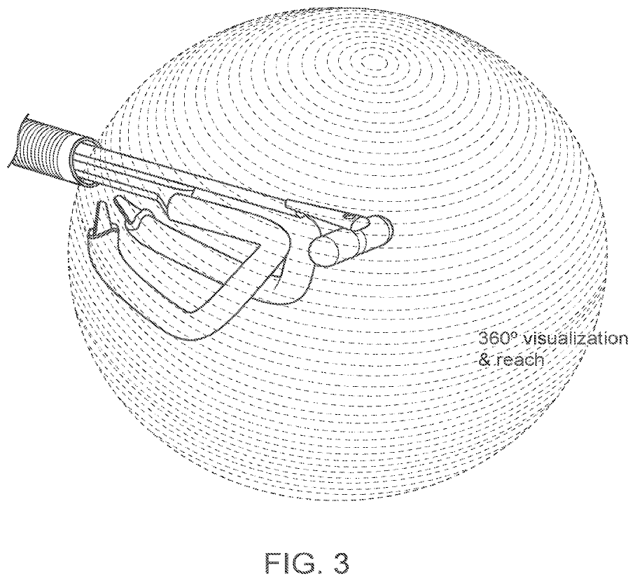

FIG. 3 shows one example of a robotic arm configured for 360-degree visualization and reach, which is able to look and operate in a sphere from a single incision. The 360-degree visualization and reach may be enabled by positioning elements and/or wrist elements of the robotic arm, which are configured to be controlled in order to provide degrees of freedom of articulation sufficient to perform surgical operations during a laparoscopic surgery.

FIGS. 4A-4C show one example of a robotic arm configured to operate front and side-to-side, as illustrated by a right-facing operating configuration (FIG. 4A), a front-facing operating configuration (FIG. 4B), and a left-facing operating configuration (FIG. 4C). In some embodiments, the robotic arm comprises positioning elements and/or wrist elements configured to be controlled in order to provide degrees of freedom of articulation sufficient to perform surgical operations during a laparoscopic surgery. In some embodiments, the robotic arm is configured for 360-degree visualization and reach, which is able to look and operate in a sphere from a single incision.

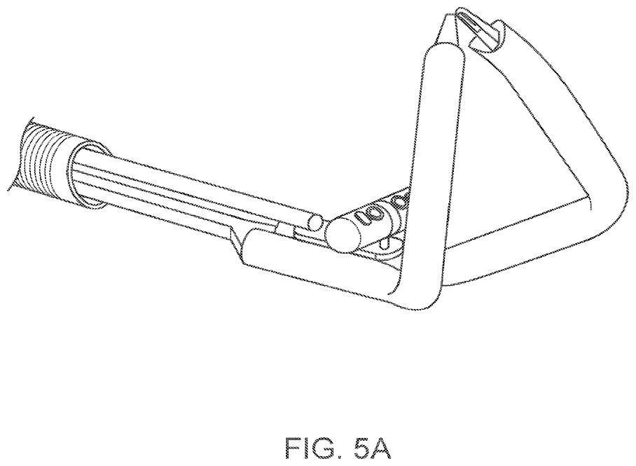

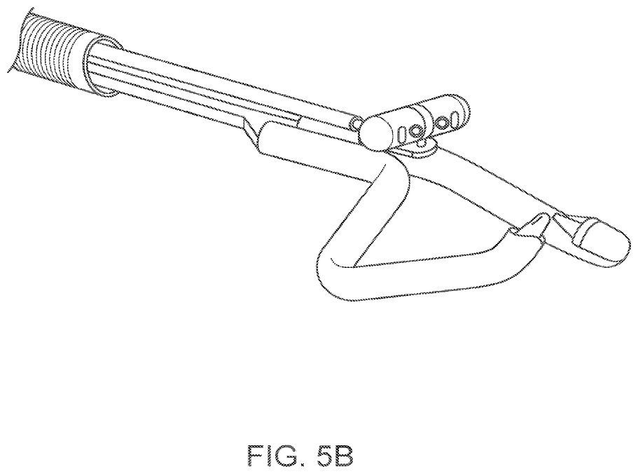

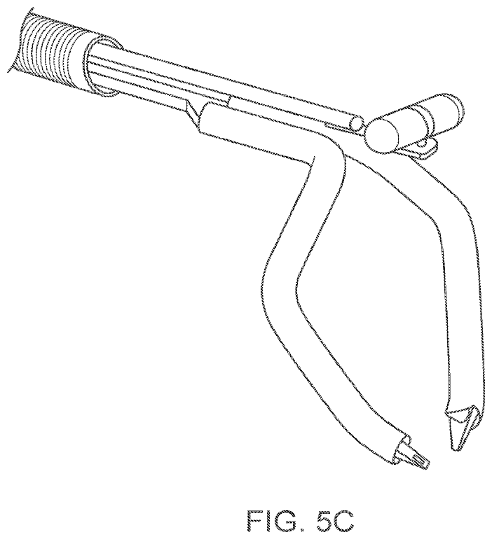

FIGS. 5A-5C show one example of a robotic arm configured to operate front, up, down, and backwards, as illustrated by an up-facing operating configuration (FIG. 5A), a front-facing operating configuration (FIG. 5B), and a down-facing operating configuration (FIG. 5C). In some embodiments, the robotic arm comprises positioning elements and/or wrist elements configured to be controlled in order to provide degrees of freedom of articulation sufficient to perform surgical operations during a laparoscopic surgery. In some embodiments, the robotic arm is configured for 360-degree visualization and reach, which is able to look and operate in a sphere from a single incision

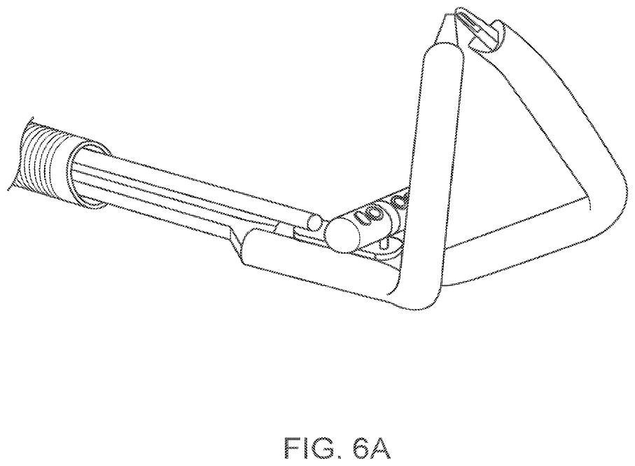

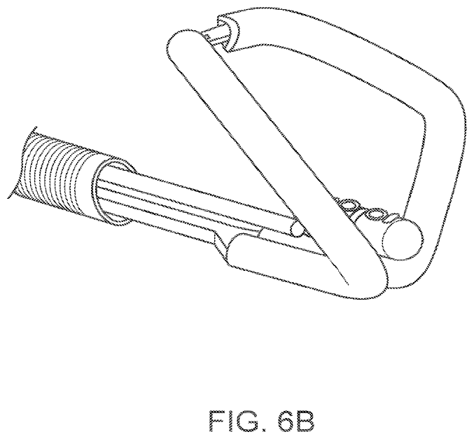

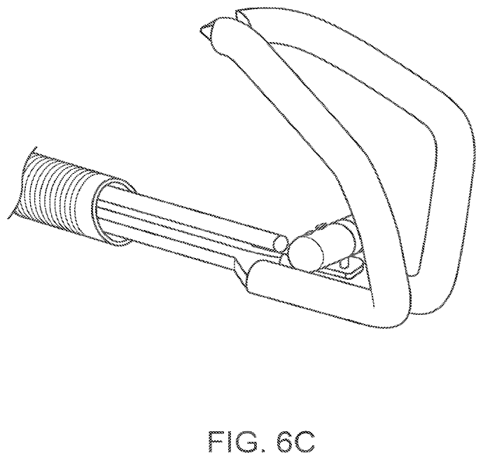

FIGS. 6A-6C show one example of a robotic arm configured to operate up and backwards, as illustrated by an up-facing operating configuration (FIG. 6A), a transition from an up-facing to a backwards-facing operating configuration (FIG. 6B), and a backwards-facing operating configuration (FIG. 6C). In some embodiments, the robotic arm comprises positioning elements and/or wrist elements configured to be controlled in order to provide degrees of freedom of articulation sufficient to perform surgical operations during a laparoscopic surgery. In some embodiments, the robotic arm is configured for 360-degree visualization and reach, which is able to look and operate in a sphere from a single incision.

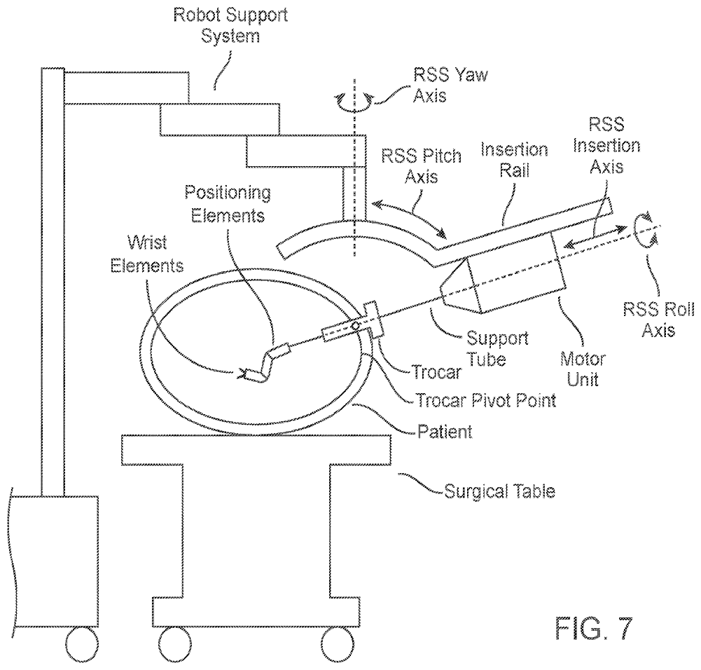

FIG. 7 shows one example of a robot support system (RSS) comprising axes and translating positioning elements about an abdomen of a subject (e.g., patient) supported by a surgical table. The RSS may facilitate the positioning and insertion of a robotic arm (e.g., comprising a support tube, a trocar, positioning elements, and wrist elements) into a body cavity (e.g., abdomen) of a subject (e.g., a patient) at a trocar pivot point during a laparoscopic surgery. The RSS may comprise a motor unit configured to control movement of the support tube (e.g., along an RSS insertion axis and/or RSS roll axis) in order to insert the robotic arm into the abdomen of the patient during laparoscopic surgery. The insertion may be guided by an insertion rail, whose movement may be controlled (e.g., along an RSS pitch axis and/or an RSS yaw axis) by the RSS.

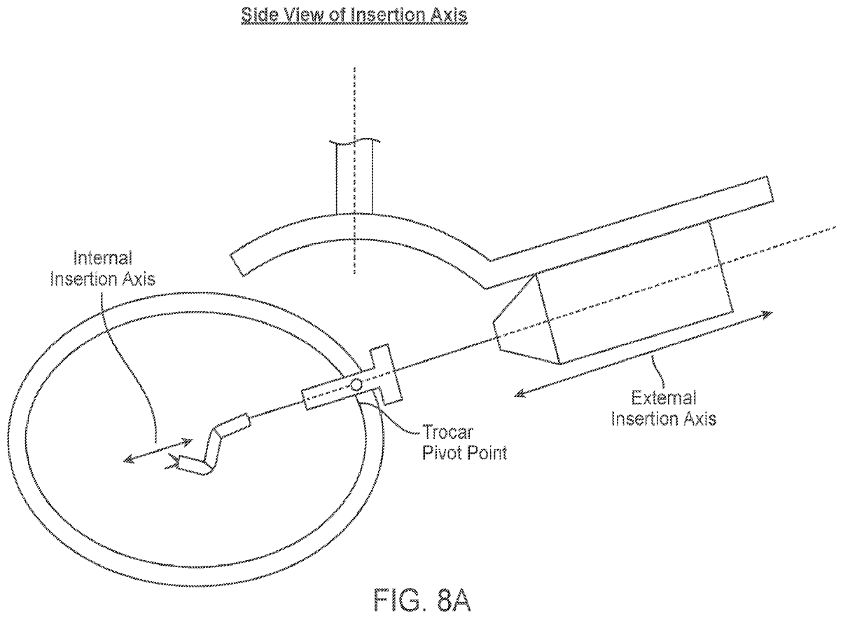

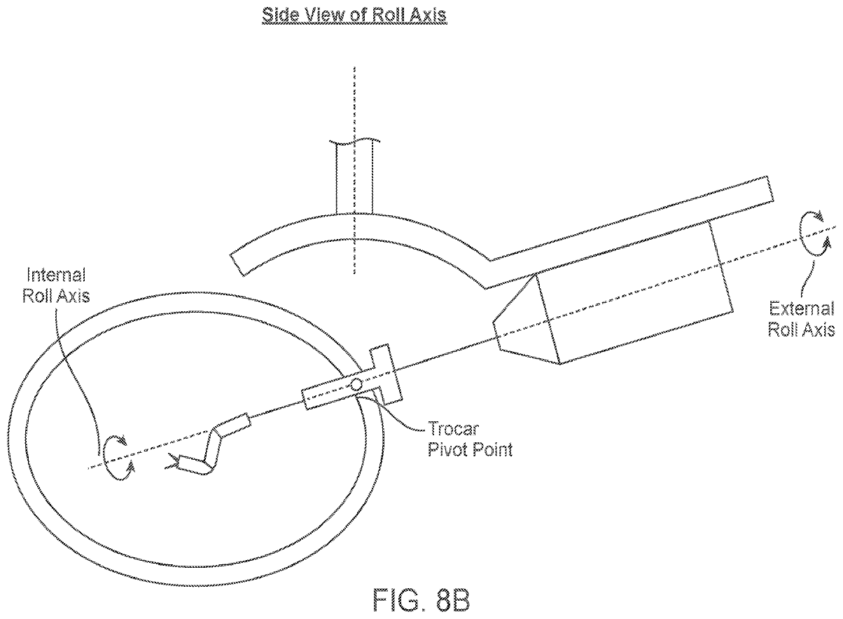

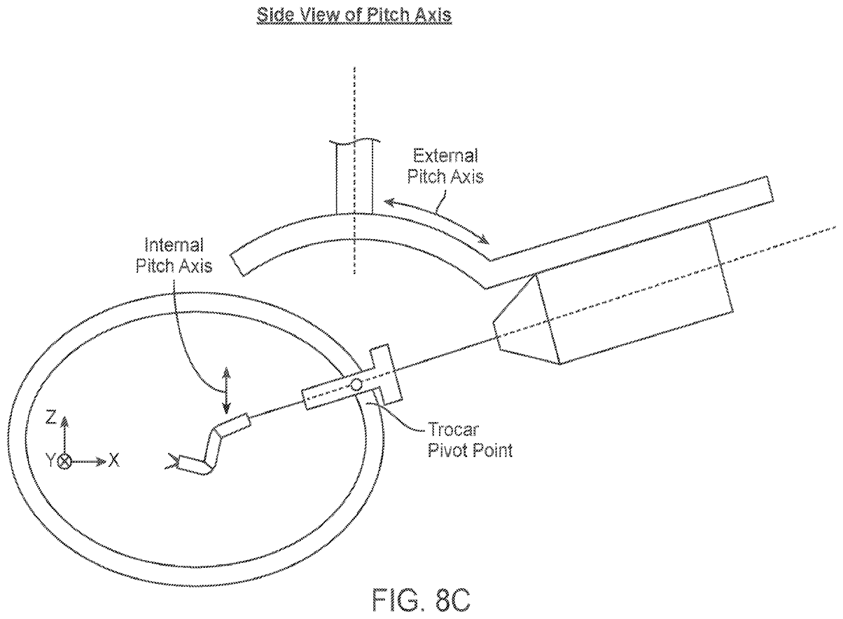

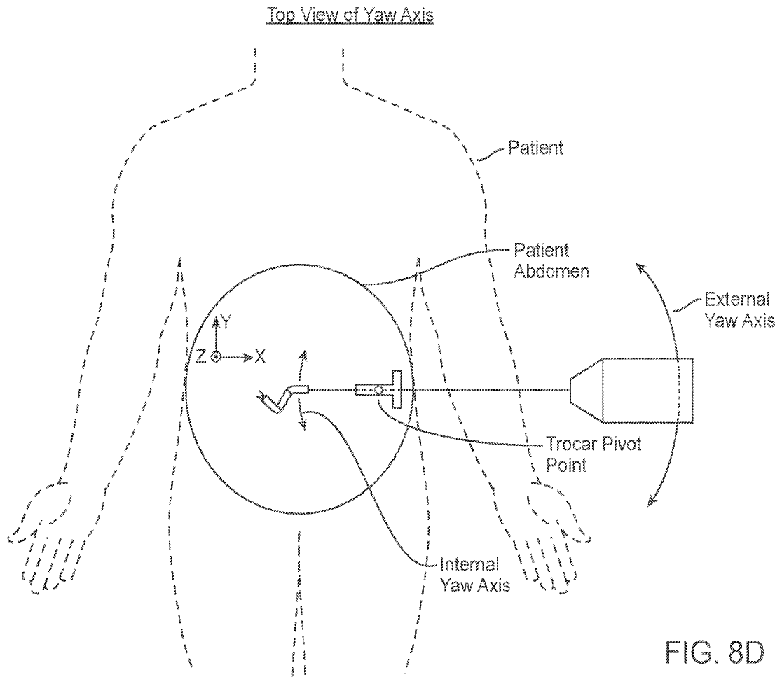

FIGS. 8A-8D show one example of various axes of the RSS, including a side view of an insertion axis (FIG. 8A), a side view of a roll axis (FIG. 8B), a side view of a pitch axis (FIG. 8C), and a top view of a yaw axis (FIG. 8D). FIG. 8A provides a side view of an insertion axis, including an internal insertion axis (inside the body cavity of the subject) and an external insertion axis (outside the body cavity of the subject). FIG. 8B provides a side view of a roll axis, including an internal roll axis (inside the body cavity of the subject) and an external roll axis (outside the body cavity of the subject). FIG. 8C provides a side view of a pitch axis, including an internal pitch axis (inside the body cavity of the subject) and an external pitch axis (outside the body cavity of the subject). FIG. 8D provides a top view of a yaw axis, including an internal yaw axis (inside the body cavity of the subject) and an external yaw axis (outside the body cavity of the subject).

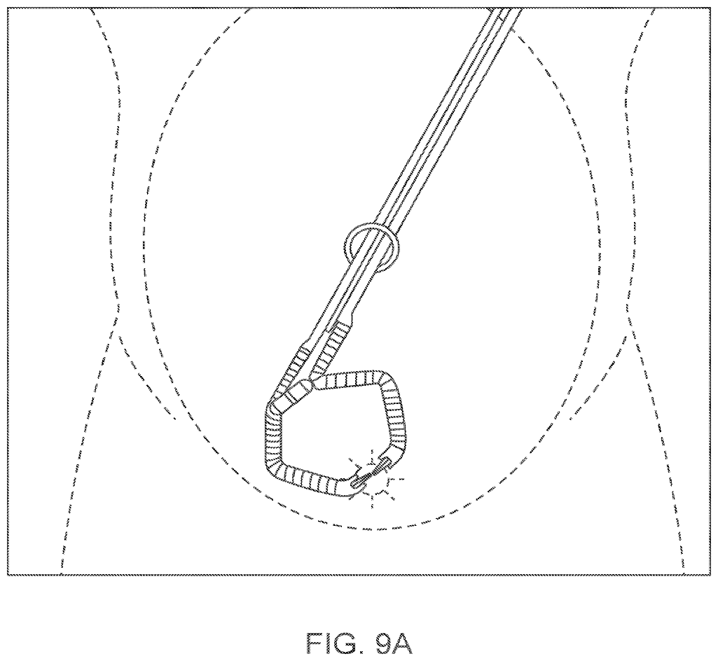

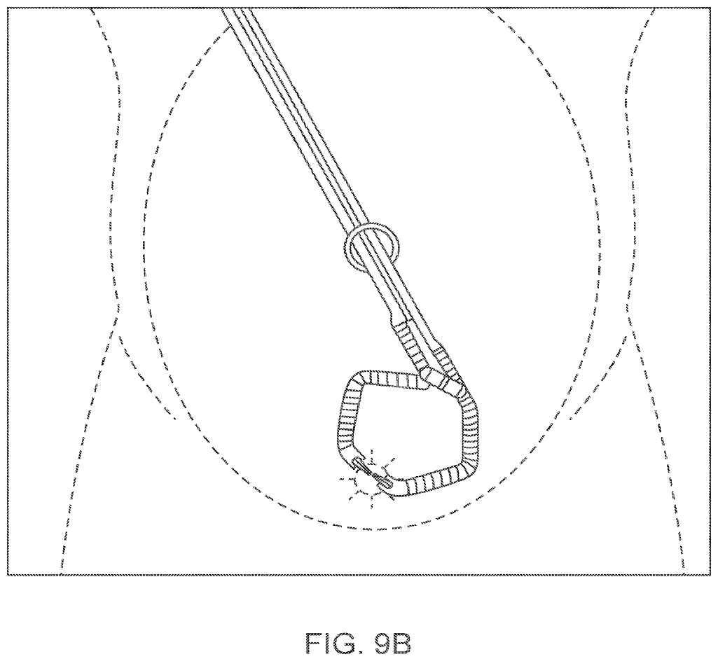

FIGS. 9A-9B show one example of using a robotic arm to approach a surgical target from two sides (left side: FIG. 9A; right side: FIG. 9B) by adjusting the yaw and insertion axes as well as relative positions of positioning elements. These figures illustrate front and side facing views of the robotic arm relative to a trocar. The robotic arm may be configured for 360-degree visualization and reach, which is able to look and operate in a sphere from a single incision. The 360-degree visualization and reach may be enabled by positioning elements and/or wrist elements of the robotic arm, which are configured to be controlled in order to provide degrees of freedom of articulation sufficient to perform surgical operations during a laparoscopic surgery.

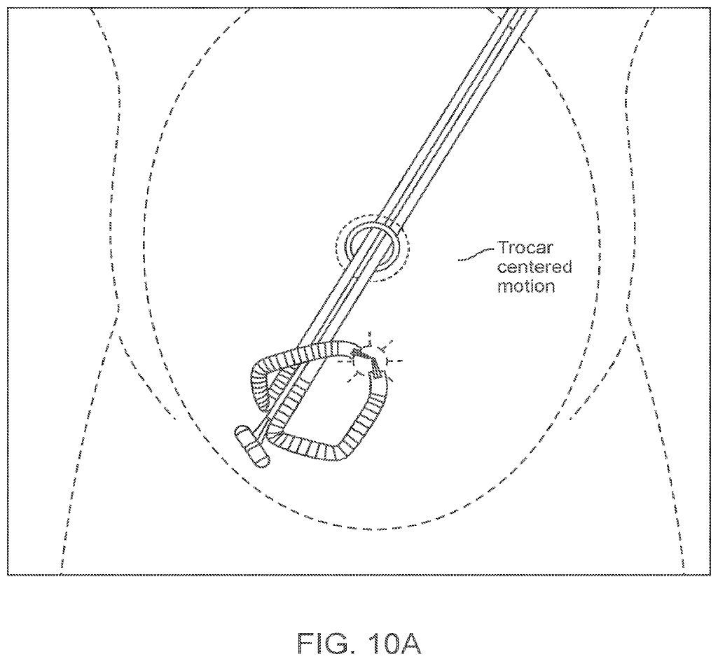

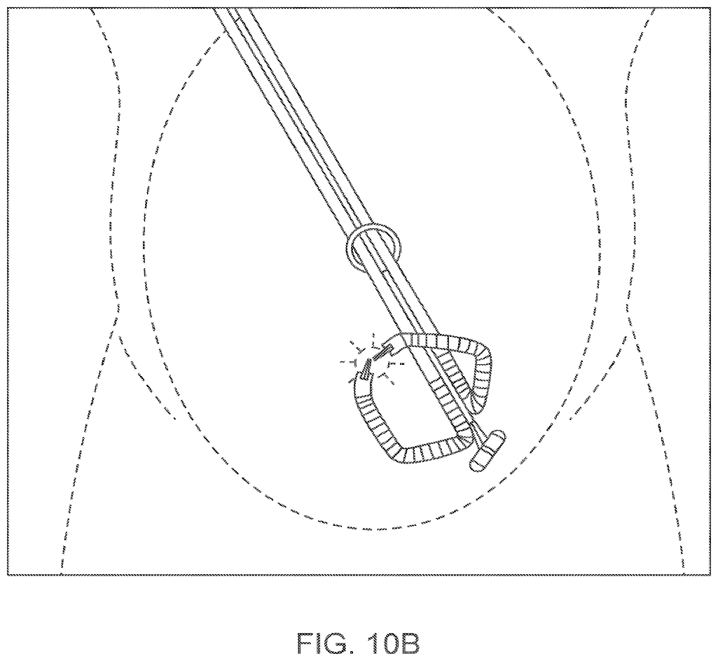

FIGS. 10A-10B show one example of using a robotic arm to approach a surgical target from two sides (left side: FIG. 10A; right side: FIG. 10B) by adjusting the yaw and insertion axes as well as relative positions of positioning elements. These figures illustrate backwards-facing views (toward an incision) of the robotic arm relative to a trocar. The robotic arm may be configured for 360-degree visualization and reach, which is able to look and operate in a sphere from a single incision. The 360-degree visualization and reach may be enabled by positioning elements and/or wrist elements of the robotic arm, which are configured to be controlled in order to provide degrees of freedom of articulation sufficient to perform surgical operations during a laparoscopic surgery.

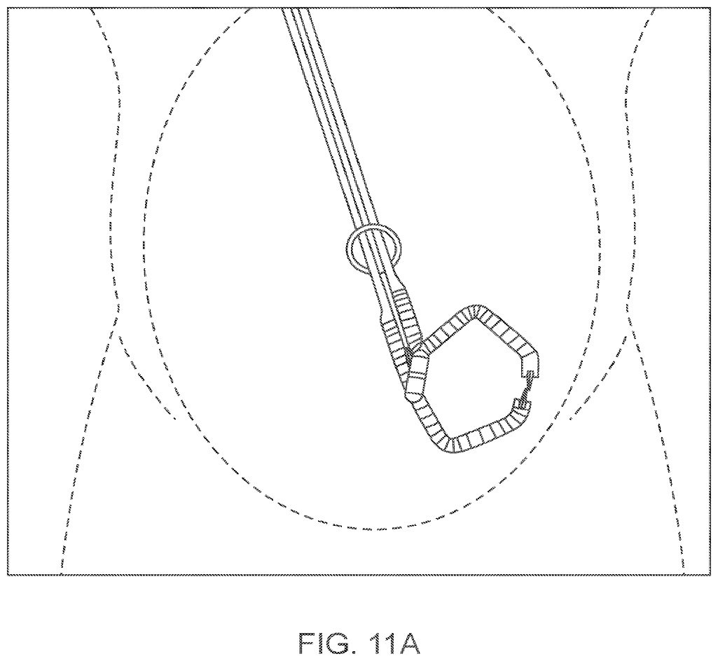

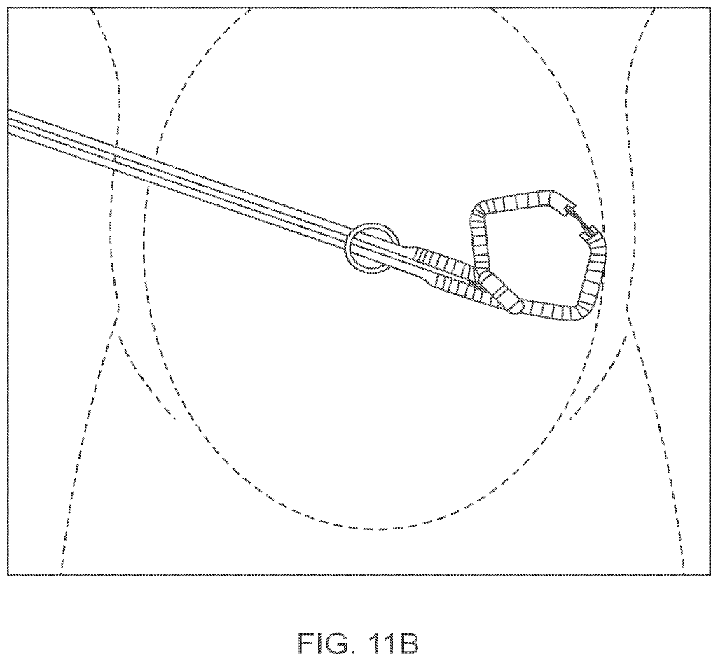

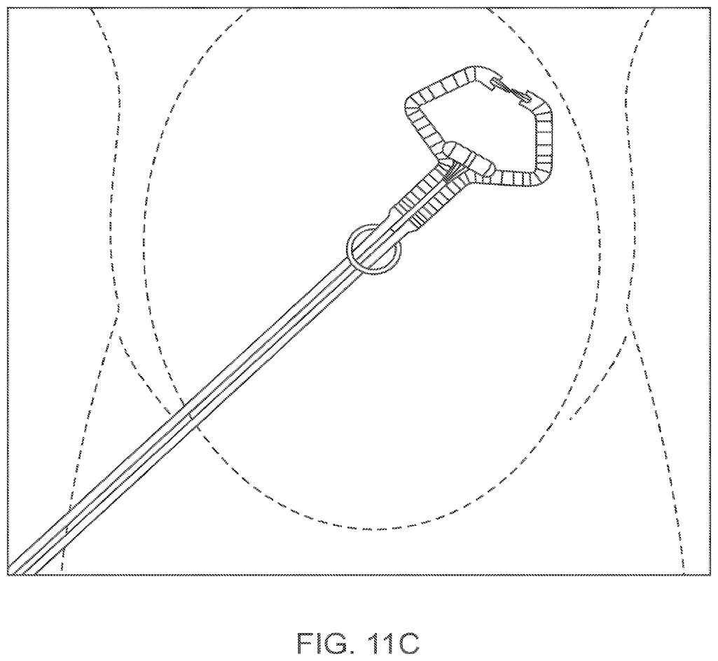

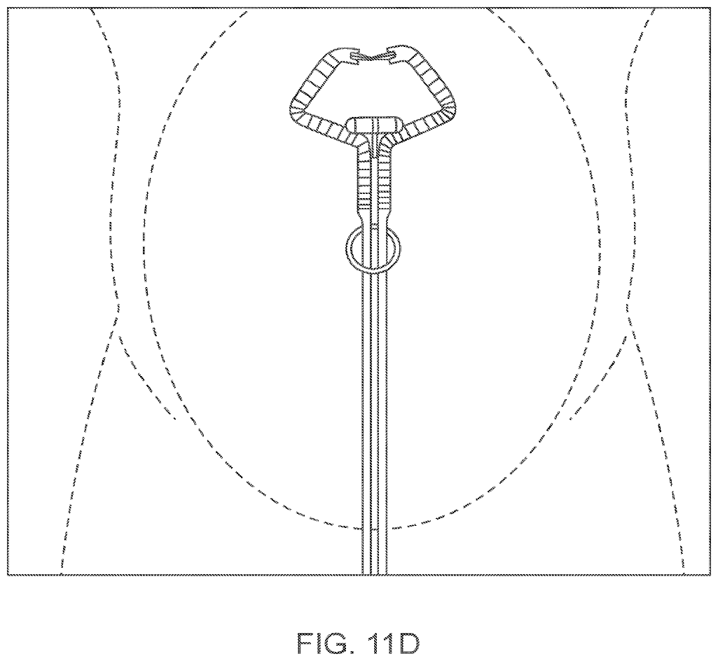

FIGS. 11A-11D show one example of using a robotic arm to traverse an abdomen of a subject from a single incision, by adjusting the yaw and insertion axes as well as relative positions of some of the positioning elements. These figures illustrate front and side facing views of the robotic arm relative to a trocar. The robotic arm may be configured for 360-degree visualization and reach, which is able to look and operate in a sphere from a single incision. The 360-degree visualization and reach may be enabled by positioning elements and/or wrist elements of the robotic arm, which are configured to be controlled in order to provide degrees of freedom of articulation sufficient to perform surgical operations during a laparoscopic surgery.

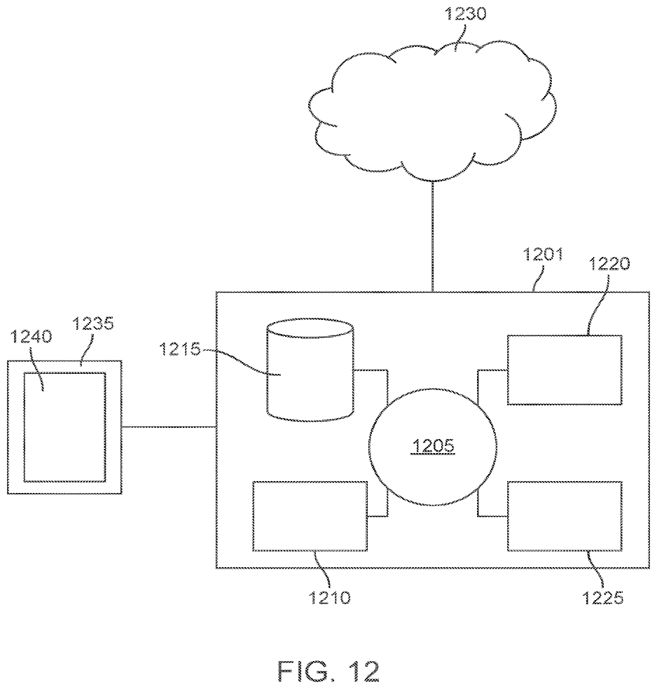

FIG. 12 shows one example of a computer system 1201 that is programmed or otherwise configured to direct operation of a device or system as described herein, including movement of components of the device or system and/or performing a surgical procedure using the device or system. The computer system 1201 regulates various aspects of systems, methods, and media of the present disclosure, such as, for example, (a) movement of one or more device or system components, (b) operation of one or more positioning elements, one or more wrist elements, and/or one or more cameras (c) adjustment of one or more parameters of one or more device or system components (e.g., one or more positioning elements, one or more wrist elements, and/or one or more cameras), (d) computational evaluation of one or more measurements of a device or system, and (e) display of various parameters including input parameters, results of a measurement, or any combination of any of these.

In some embodiments, a computer system 1201 is an electronic device of a user (e.g. smartphone, laptop) or, in some embodiments, is remotely located with respect to the electronic device. The electronic device, in some embodiments, is a mobile electronic device.

The computer system 1201 includes a central processing unit (CPU, also “processor” and “computer processor” herein) 1205, which, in some embodiments, is a single core or multi core processor, or a plurality of processors for parallel processing. The computer system 1201 also includes memory or memory location 1210 (e.g., random-access memory, read-only memory, flash memory), electronic storage unit 1215 (e.g., hard disk), communication interface 1220 (e.g., network adapter) for communicating with one or more other systems, and peripheral devices 1225, such as cache, other memory, data storage and/or electronic display adapters. The memory 1210, storage unit 1215, interface 1220 and peripheral devices 1225 are in communication with the CPU 1205 through a communication bus (solid lines), such as a motherboard. The storage unit 1215 is configured as a data storage unit (or data repository) for storing data. The computer system 1201 is operatively coupled to a computer network (“network”) 1230 with the aid of the communication interface 1220. The network 1230 is the Internet, an internet and/or extranet, or an intranet and/or extranet that is in communication with the Internet. The network 1230 in some embodiments is a telecommunication and/or data network. The network 1230 includes one or more computer servers, which enable distributed computing, such as cloud computing. The network 1230, in some embodiments, with the aid of the computer system 1201, implements a peer-to-peer network, which enables devices coupled to the computer system 1201 to behave as a client or a server.

The CPU 1205 is configured to execute a sequence of machine-readable instructions, which are be embodied in a program or software. The instructions are stored in a memory location, such as the memory 1210. The instructions are directed to the CPU 1205, which is subsequently program or otherwise configure the CPU 1205 to implement methods of the present disclosure. Examples of operations performed by the CPU 1205 include fetch, decode, execute, and writeback.

The CPU 1205 is part of a circuit, such as an integrated circuit. One or more other components of the system 1201 are included in the circuit. In some embodiments, the circuit is an application specific integrated circuit (ASIC).

The storage unit 1215 stores files, such as drivers, libraries and saved programs. The storage unit 1215 stores user data, e.g., user preferences and user programs. The computer system 1201 in some embodiments include one or more additional data storage units that are external to the computer system 1201, such as located on a remote server that is in communication with the computer system 1201 through an intranet or the Internet.

The computer system 1201 communicates with one or more remote computer systems through the network 1230. For instance, the computer system 1201 communicates with a remote computer system of a user (e.g., a second computer system, a server, a smart phone, an iPad, or any combination thereof). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user accesses the computer system 1201 via the network 1230.

Methods as described herein are implemented by way of machine (e.g., computer processor) executable code stored on an electronic storage location of the computer system 1201, such as, for example, on the memory 1210 or electronic storage unit 1215. The machine executable or machine readable code is provided in the form of software. During use, the code is executed by the processor 1205. In some embodiments, the code is retrieved from the storage unit 1215 and stored on the memory 1210 for ready access by the processor 1205. In some situations, the electronic storage unit 1215 is precluded, and machine-executable instructions are stored on memory 1210.

A machine readable medium, such as computer-executable code, takes many forms, including but not limited to, a tangible storage medium, a carrier wave medium or physical transmission medium. Non-volatile storage media include, for example, optical or magnetic disks, such as any of the storage devices in any computer(s) or the like, such as is used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media takes the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer reads programming code and/or data. Many of these forms of computer readable media is involved in carrying one or more sequences of one or more instructions to a processor for execution.

The computer system 1201, in some embodiments, includes or is in communication with an electronic display 1235 that comprises a user interface (UI) 1240 for providing, for example, a graphical representation or other visualization (e.g., image data or video data) of operation of the robotic arm during laparoscopic surgery, one or more parameters that are input or adjusted by a user or by a controller, or any combination thereof. Examples of UI's include, without limitation, a graphical user interface (GUI) and web-based user interface.

Methods and systems of the present disclosure are, in some embodiments, implemented by way of one or more algorithms. An algorithm, in some embodiments, is implemented by way of software upon execution by the central processing unit 1205. The algorithm may, for example, regulate various aspects of systems, methods, and media of the present disclosure, such as, for example, (a) movement of one or more device or system components, (b) operation of one or more positioning elements, one or more wrist elements, and/or one or more cameras (c) adjustment of one or more parameters of one or more device or system components (e.g., one or more positioning elements, one or more wrist elements, and/or one or more cameras), (d) computational evaluation of one or more measurements of a device or system, and (e) display of various parameters including input parameters, results of a measurement, or any combination of any of these.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein is employed in practicing the invention. It is intended that the following claims define the scope of the invention and that methods and structures within the scope of these claims and their equivalents be covered thereby.

1. A system for performing laparoscopic surgery, comprising:
a set of robotic arms, each of the set of robotic arms comprising at least one end-effector, and at least one camera,
wherein the at least one internal end-effector and the at least one camera have sufficient degrees of freedom of adjustment of position and sufficient degrees of freedom of adjustment of orientation to provide a full range of motion and orientation of operation and view perspective for performing the laparoscopic surgery while inserted into a body cavity of a subject.

2. The system of claim 1, wherein the body cavity is an abdomen of the subject.

3. The system of claim 1, wherein the full range of motion and orientation of operation and view perspective comprises a front-facing, back-facing, side-facing, up-facing, down-facing, left-facing, or right-facing direction of motion or orientation of operation and view perspective.

4. The system of claim 1, wherein the full range of motion and orientation of operation and view perspective comprises ability to be adjusted by 90 degrees between any two positions or directions of motion or orientation of operation and view perspective.

5. The system of claim 1, wherein the set of robotic arms and/or the at least one camera comprise external degrees of freedom that enable internal degrees of freedom to be translated about the body cavity of the subject.

6. The system of claim 1, wherein each of the set of robotic arms comprises:
i. a first shaft having a first axis of symmetry;
ii. a second shaft having a second axis of symmetry;
iii. a third shaft having a third axis of symmetry;
iv. an end effector for insertion into a body cavity of a subject to perform a laparoscopic surgical operation therein;
v. a first actuator rotating the second shaft with respect to the first shaft about a first primary axis;
vi. a second actuator rotating the second shaft with respect to the second shaft about a second primary axis; and (vii) a third actuator rotating the end effector with respect to the third shaft about a third primary axis.

7. The system of claim 1, wherein the set of robotic arms comprises at least two robotic arms.

8. The system of claim 1, wherein the set of robotic arms provides at least one degree of freedom of articulation that is internal to the body cavity of the subject during the laparoscopic surgical operation.

9. The system of claim 8, wherein the set of robotic arms provides at least 2, 3, 4, 5, 6, 7, 8, 9, 10, 11, 12, or more than 12 degrees of freedom of articulation that are internal to the body cavity of the subject during the laparoscopic surgical operation.

10. The system of claim 1, further comprising a camera positioning arm, and a camera actuator rotating the at least one camera with respect to the camera positioning arm about a primary camera axis.

11. The system of claim 1, wherein the at least one camera is configured to provide at least one degree of freedom of articulation that is internal to the body cavity of the subject during the laparoscopic surgical operation.

12. The system of claim 11, wherein the at least one camera is configured to provide at least 2, 3, 4, 5, 6, 7, 8, 9, 10, 11, 12, or more than 12 degrees of freedom of articulation that are internal to the body cavity of the subject during the laparoscopic surgical operation.

13. The system of claim 1, wherein the at least one camera comprises at least one stereoscopic camera.

14. The system of claim 13, wherein the at least one stereoscopic camera comprises at least one actuatable stereoscopic camera.

15. The system of claim 1, wherein the system provides at least 2, 3, 4, 5, 6, 7, 8, 9, 10, 11, 12, or more than 12 degrees of freedom of articulation that are internal to the body cavity of the subject selected from: an insertion degree of freedom, a roll degree of freedom, a pitch degree of freedom, and a yaw degree of freedom.

16. The system of claim 1, wherein the at least one camera comprises a flash, a lens, a flash, a light, or any combination thereof.

17. The system of claim 1, wherein the at least one camera comprises a stereoscopic camera, an infrared camera, an optical camera, or any combination thereof.

18. The system of claim 1, wherein the end effector is a pincer, a grasper, a needle driver, a forceps, or any combination thereof.

19. The system of claim 10, wherein the camera actuator further rotates the camera with respect to the camera positioning arm about a secondary camera axis that is perpendicular to the primary camera axis.

20. The system of claim 10, wherein the primary camera axis and the secondary camera axis are perpendicular to an axis of symmetry of the camera positioning arm.

21. The system of claim 6, wherein the first actuator further rotates the second shaft with respect to the first shaft about a first secondary axis perpendicular to the first primary axis.

22. The system of claim 6, wherein the second actuator further rotates the third shaft with respect to the second shaft about a second secondary axis perpendicular to the second primary axis.

23. The system of claim 6, wherein the third actuator further rotates the end effector with respect to the third shaft about a third secondary axis perpendicular to the third primary axis.

24. The system of claim 6, further comprising a trocar, and wherein the camera positioning arm, the first shaft of one or more of the two or more arms, or both are translatable with respect to the trocar.

25. A method for performing laparoscopic surgery, comprising inserting at least a portion of a set of robotic arms into a body cavity of a subject to perform a laparoscopic surgical operation therein, each of the set of robotic arms comprising at least one end-effector, and at least one camera, wherein the at least one internal end-effector and the at least one camera have sufficient degrees of freedom of adjustment of position and sufficient degrees of freedom of adjustment of orientation to provide a full range of motion and orientation of operation and view perspective for performing the laparoscopic surgery while inserted into a body cavity of a subject.

26. The method of claim 25, wherein the body cavity is an abdomen of the subject.

27. The method of claim 25, wherein the full range of motion and orientation of operation and view perspective comprises a front-facing, back-facing, side-facing, up-facing, down-facing, left-facing, or right-facing direction of motion or orientation of operation and view perspective.

28. The method of claim 25, wherein the full range of motion and orientation of operation and view perspective comprises ability to be adjusted by 90 degrees between any two positions or directions of motion or orientation of operation and view perspective.

29. The method of claim 25, wherein the set of robotic arms and/or the at least one camera comprise external degrees of freedom that enable internal degrees of freedom to be translated about the body cavity of the subject.

30. The method of claim 25, wherein each of the set of robotic arms comprises:
(i) a first shaft having a first axis of symmetry;
(ii) a second shaft having a second axis of symmetry;
(iii) a third shaft having a third axis of symmetry;
(iv) an end effector for insertion into a body cavity of a subject to perform a laparoscopic surgical operation therein;
(v) a first actuator rotating the second shaft with respect to the first shaft about a first primary axis;
(vi) a second actuator rotating the second shaft with respect to the second shaft about a second primary axis; and
(vii) a third actuator rotating the end effector with respect to the third shaft about a third primary axis.

31. The method of claim 25, wherein the set of robotic arms comprises at least two robotic arms.

32. The method of claim 25, wherein the set of robotic arms provides at least one degree of freedom of articulation that is internal to the body cavity of the subject during the laparoscopic surgical operation.

33. The method of claim 32, wherein the set of robotic arms provides at least 2, 3, 4, 5, 6, 7, 8, 9, 10, 11, 12, or more than 12 degrees of freedom of articulation that are internal to the body cavity of the subject during the laparoscopic surgical operation.

34. The method of claim 25, wherein the set of robotic arms further comprises a camera positioning arm, and a camera actuator rotating the at least one camera with respect to the camera positioning arm about a primary camera axis.

35. The method of claim 25, wherein the at least one camera is configured to provide at least one degree of freedom of articulation that is internal to the body cavity of the subject during the laparoscopic surgical operation.

36. The method of claim 35, wherein the at least one camera is configured to provide at least 2, 3, 4, 5, 6, 7, 8, 9, 10, 11, 12, or more than 12 degrees of freedom of articulation that are internal to the body cavity of the subject during the laparoscopic surgical operation.

37. The method of claim 25, wherein the at least one camera comprises at least one stereoscopic camera.

38. The method of claim 37, wherein the at least one stereoscopic camera comprises at least one actuatable stereoscopic camera.

39. The method of claim 25, wherein the system provides at least 2, 3, 4, 5, 6, 7, 8, 9, 10, 11, 12, or more than 12 degrees of freedom of articulation that are internal to the body cavity of the subject selected from: an insertion degree of freedom, a roll degree of freedom, a pitch degree of freedom, and a yaw degree of freedom.

40. The method of claim 25, wherein the camera comprises a flash, a lens, a flash, a light, or any combination thereof.

41. The method of claim 25, wherein the camera comprises a stereoscopic camera, an infrared camera, an optical camera, or any combination thereof.

42. The method of claim 25, wherein the end effector is a pincer, a grasper, a needle driver, a forceps, or any combination thereof.

43. The method of claim 34, wherein the camera actuator further rotates the camera with respect to the camera positioning arm about a secondary camera axis that is perpendicular to the primary camera axis.

44. The method of claim 34, wherein the primary camera axis and the secondary camera axis are perpendicular to an axis of symmetry of the camera positioning arm.

45. The method of claim 30, wherein the first actuator further rotates the second shaft with respect to the first shaft about a first secondary axis perpendicular to the first primary axis.

46. The method of claim 30, wherein the second actuator further rotates the third shaft with respect to the second shaft about a second secondary axis perpendicular to the second primary axis.

47. The method of claim 30, wherein the third actuator further rotates the end effector with respect to the third shaft about a third secondary axis perpendicular to the third primary axis.

48. The method of claim 30, wherein the set of robotic arms further comprises a trocar, and wherein the camera positioning arm, the first shaft of one or more of the two or more arms, or both are translatable with respect to the trocar.

49. A platform comprising:
(a) the system of claim 1;
(b) a motor providing power to the system; and
(c) a gantry coupled to the motor.

50. The platform of claim 49, wherein the motor provides power to one or more of
(a) a camera actuator;
(b) a first actuator;
(c) a second actuator; and
(d) a third actuator.

51. The platform of claim 49, wherein the gantry couples to the motor by one or more of a rotatable coupling and a translatable coupling.

52. The platform of claim 49, further comprising a surgical table.

53. The platform of claim 49, further comprising a display receiving an image from the camera.

54. The platform of claim 53, wherein the display is a head-mounted display.

55. The platform of claim 49, further comprising an input providing an actuation command to the motor.
